(19) United States
(12) Patent Application Publication
(10) Pub. No.: US 2012/0253078 A1
(43) Pub. Date: Oct. 4, 2012

(54) Agent for Improving Carcass Performance in Finishing Hogs

(75) Inventors: Patrick Monahan, Columbia, MD (US); Stephen Hiu, Columbia, MD (US)

(73) Assignee: IGENE BIOTECHNOLOGY, INC., Columbia, MD (US)

(21) Appl. No.: 13/525,321

(22) Filed: Jun. 17, 2012

Related U.S. Application Data
(62) Division of application No. 12/741,373, filed on May 4, 2010.
(60) Provisional application No. 60/986,935, filed on Nov. 9, 2007.

(51) Int. Cl. C07C 49/825

(52) U.S. Cl. 568/331

(57) ABSTRACT

A food supplement for finishing hogs containing astaxanthin is described.

### FIELD OF THE INVENTION

The present invention relates to an agent that improves carcass characteristics of late finishing pigs destined for food use. More specifically, the present invention relates to the addition of an astaxanthin in the diet of finishing pigs to improve overall growth performance, such as, to decrease backfat accumulation, to decrease backfat depth, to improve percent of standardized fat-free lean, and to improve color measurements in the carcass of finishing pigs.

### BACKGROUND OF THE INVENTION

The production of swine (also known as pigs or hogs) for food use is an important industry, for example, in the United States, more than 100 million pigs are produced each year. Because margins are low, there is demand among producers for agents that increase productivity, such as by increasing the proportion of meat per carcass, increasing loin eye area, decreasing the amount of backfat, or which provide other beneficial characteristics, such as improved meat color measurements.

The present invention now provides such an agent, which consists of astaxanthin preferably from a natural source, such as Phaffia rhodozyma yeast.

Astaxanthin (3,3′-dihydroxy-β,β-carotene-4,4′dione) is the carotenoid that imparts a pink to orange to red coloration to eggs, flesh, filets, and skin of shrimp, salmon and trout. Astaxanthin belongs to a group of pigments known as carotenoids, which exhibit antioxidant properties. Few animals synthesize astaxanthin, with most relying on food intake as the source of astaxanthin. Crustaceans, such as crabs, lobsters, crawfish and shrimp, obtain the characteristic hue of the carapace and flesh from astaxanthin obtained from food in the environment, whether farm-raised or harvested in the wild. Although astaxanthin has been known to color the flesh of salmon and trout, as this compound is a normal part of their diet, it has not previously been known that astaxanthin has the ability to affect color characteristics of the meat of hogs/swine/pigs as described herein.

Astaxanthin can be synthesized or obtained from natural sources, such as yeast, such as Phaffia rhodozyma (Xanthophyllomyces dendrorhous) mentioned above, from certain algae, such as Hematococcus pluvialis, or from certain bacteria, such as certain Paracoccus spp.

Johnson et al. reported that the astaxanthin from broken yeast, Phaffia rhodozyma, was deposited in the egg yolks of chickens fed over a 16-day regime (1980. Poultry. Sci. 59:1777-1782). Studies also have shown that the presence of astaxanthin in chicken feed increased fertility, weight gain and feed utilization (Yang et al., 2006. Asian-Aust. J. Anim. Sci. 19(7):1-7). In addition, Phaffia rhodozyma is commonly used, and is commercially available as a source of astaxanthin for the pigmentation of farm-raised salmonids (Johnson and An, 1991. Crit. Rev. Biotechnology. 11(4):297-326).

Certain strains of Phaffia have been manipulated to contain and to produce enhanced levels of astaxanthin, see, for example, U.S. Pat. Nos. 5,182,208 and 5,356,809. Thus, certain strains of Phaffia contain more than 500 parts per million (ppm) astaxanthin per gram dry weight of yeast, more than 1000 ppm, more than 1500 ppm, more than 2000 ppm, more than 2500 ppm, more than 3000 ppm, more than 3500 ppm, more than 4000 ppm, more than 4500 ppm, more than 5000 ppm, more than 5500 ppm, more than 6000 ppm, more than 6500 ppm, more than 7000 ppm, more than 7500 ppm, more than 8000 ppm, more than 9000 ppm, more than 10,000 ppm, 11,000 ppm, more than 12,000 ppm, more than 13,000 ppm, more than 14,000 ppm, more than 15,000 ppm or more astaxanthin per dry gram of yeast. Sources providing greater amounts of astaxanthin provide an economic advantage.

Dietary carotenoids, including astaxanthin, are used as a nutraceutical supplement. Dietary carotenoids are asserted to prevent infections. Astaxanthin, a nonprovitamin α-carotenoid is as active and at times more active than β-carotene in enhancing immune response in animals and humans (Chew and Park, 2004. J. Nutr. 134:257S-261S). Astaxanthin has also been described in a composition comprising a source of long chain polyunsaturated fatty acid and a carotenoid, for prophylactic and/or therapeutic use in the healing of trauma-induced and stress-induced inflammatory conditions (PCT Pub. No. WO2004/112776).

For animal use, astaxanthin was used as a feed ingredient in combination with L-ascorbic acid derivatives as an anti-stress composition for animals (U.S. Pat. No. 5,937,790). In another application, an agent, astaxanthin, is claimed to suppress body fat gain in mammals where the mammal is a human, based on a study done in mice fed high doses of astaxanthin as part of a high-fat diet (U.S. Publ. No. 2007129436).

In the swine production industry, astaxanthin from astaxanthin-rich algae meal is alleged to improve sow and litter performance when added as an ingredient incorporated into normal diet when used in a feed before farrowing, during lactation and after weaning (Inborr et al., 1997. Proceedings of the 7^{th} International Symposium on Digestive Physiology in Pigs, 26-28 May). The mechanism for the improvement was thought to be the result of an improvement in immune response of the animals fed astaxanthin. U.S. Pat. No. 6,054,491 teaches addition of 5 ppm astaxanthin in the feed improved performance of sows by reducing the number of stillborn in the litter and improving parity. Yang et al. showed that addition of 0, 1.5 and 3.0 ppm astaxanthin incorporated into the diet of finishing pigs had no significant effect on production performance, but that there was a linear effect on dressing percentage, backfat thickness and loin muscle area with increasing dietary astaxanthin level (Yang et al., 2006. Asian-Aust. J. Anim. Sci. 19(7):1-7).

In contrast, the invention described herein shows that increasing concentrations of astaxanthin did not result in linear improvements in carcass traits and meat quality. Surprisingly, there is an optimum range for effectiveness that peaks in the range of 5 to 10 ppm so that incorporation of 20 ppm astaxanthin in the diet, for example, showed no additional benefit. Thus, a composition of interest contains 3.5 ppm, 4 ppm, 5 ppm, 6 ppm, 7 ppm, 8 ppm, 9 ppm, 10 ppm, 11 ppm, 12 ppm, 13 ppm, 14 ppm, 15 ppm, 16 ppm, 17 ppm, 18 ppm or 19 ppm astaxanthin. The reasons for the improved carcass traits and meat quality when astaxanthin enriched feed is used according to the present invention are not known.

### SUMMARY OF THE INVENTION

The present invention thus provides an agent for improving the carcass traits and meat quality of finishing pigs (or hogs or swine, as used in the domesticated or agricultural trade) destined for slaughter to yield food products. Any animal of the genus Sus, such as S. barbatus, S. scrofa, S. celebensis and S. domestica, that can be used as a food source, can be fed the agent of interest. In a preferred embodiment, astaxanthin provided in the diet is supplied by astaxanthin produced by Phaffia rhodozyma. The agent according to the invention may consist of a mixture of different sources of astaxanthin including synthetic astaxanthin and other naturally produced astaxanthin, such as from bacteria or algae.

### DETAILED DESCRIPTION OF THE INVENTION

In the agricultural arts, finishing pigs or hogs generally include growing females (gilts) and growing castrated males (barrows) that are being fed for slaughter. Typically, pigs are classified by weight and/or age. Hence, piglets are animals from birth to weaning, generally from 14 to 35 days of age; nursery pigs are animals from weaning to about 70 days of age; growing pigs are animals from about 70 days of age to about 125 days of age, where the animals generally weigh about 160 lbs.; and finishing pigs are animals from about 125 days of age to about 190 days of age, where generally the market weight of a hog is about 270 lbs. That is about the time the pigs reach sexual maturity.

For the purposes of the instant invention, the edible composition of interest generally is provided to pigs destined for slaughter, the finishing hogs. However, the edible composition of interest can be used for pigs of any age, any stage of development or any weight.

A suitable source of the astaxanthin used in the invention is the yeast, Phaffia rhodozyma. The astaxanthin can be isolated or retained in the yeast body, wherein the yeast is dried. Astaxanthin is commercially available under the trade name Aquasta® (Astaxanthin Partners Ltd., Decatur, Ill.), a dried yeast composition which contains a minimum of 1% astaxanthin, corresponding to a 10,000 ppm product. Production of the yeast is described in U.S. Pat. Nos. 5,356,809 and 5,182,208.

The agent can be any composition that contains a suitable amount of astaxanthin for use in the practice of the instant invention. Thus, the agent can be pure astaxanthin, a composition of pure astaxanthin and inert ingredients, a dried cell that produces astaxanthin, such as a bacteria or yeast, a composition of a dried cell containing astaxanthin and inert ingredients and so on. The composition can be solid or liquid.

The present invention further provides a method of simultaneously decreasing backfat while increasing fat-free lean percent and altering meat color in pigs. In a preferred embodiment of said method according to the invention, astaxanthin, which is obtained from Phaffia rhodozyma, is administered to the diet of pigs ideally in the range of from about 3.5 to about 10 mg agent per kg feed.

The astaxanthin can be used in a variety of ways for ingestion by hogs. Thus, the astaxanthin agent can be an ingredient in a mixture, can be part of a feed, added to a feed, a component of a supplement, can be part of a food, added to a food, dissolved in a liquid, such as a vitamin solution, a supplement, a salt solution, a sugar solution, water and so on, and the like. Suitable compositions to which an agent of interest can be added can contain at least of one a protein, a carbohydrate, a fiber source, a mineral, a vitamin or other nutrient. The composition can be liquid or dry. A suitable protein source can be any suitable for ingestion by hogs, and thus, can be of plant, yeast or animal origin.

Thus, in one embodiment, the agent of interest is admixed with a food stuff or feed for finishing hogs. Thus, the agent of interest can be admixed to form a feed, or can be admixed with a feed. In some cases, the composition of interest can be added to a food, such as a plant, yeast or animal stuff, such as table scraps, remains, portions of foods unusable for sale, portions normally discarded in the food preparation business and so on. Because the agent of interest is relatively inert, the agent of interest can be admixed with any of a variety of edible compositions, such as, food sources suitable for ingestion by a hog.

An alternative food or feed for hogs are prepared foods, such as a prepared, dry pelleted product. Such products are known in the art, sometimes known as a diet food or an animal feed, and the composition of each is a design choice. The agent of interest can be integrated into the feed during manufacturing and processing.

Alternatively, the composition of interest can be added to or mixed with the animal feed. For example, that can occur by admixing a dry astaxanthin or a dry astaxanthin-containing powder with the feed preparation. Alternatively, astaxanthin can be suspended in a liquid, such as a supplement containing a vitamin, a mineral or both, and administered to a hog, or can be admixed with a food or feed.

The invention now will be exemplified in the following non-limiting examples.

### Example

### Growth Performance and Carcass Characteristics of Pigs Fed Increasing Levels of Astaxanthin

Procedures used in the experiment were approved by the Kansas State University Animal Care and Use Committee. The project was conducted at the KSU Swine Teaching and Research Farm. Pigs were housed in an environmentally regulated finishing building with pens over a totally slatted floor that provided approximately 8 ft² per pig. Each pen was equipped with a dry self-feeder and one nipple waterer, thereby providing ad libitum access to feed and water. The facility was a mechanically ventilated room with a pull-plug manure storage pit.

Forty-eight barrows (PIC, TR4×C22 cross) averaging 215 lb were used in the study. Pigs were blocked by weight and randomly allotted to one of the four dietary treatments with two pigs per pen and six pens per treatment. Experimental diets were provided in meal form and astaxanthin (0, 5, 10 and 20 ppm) was added to the control diet at the expense of corn starch to achieve the dietary treatments (Table 1). In Table 1, CP is crude protein; P is phosphate; ME is metabolizable energy; and TID is true ileal digestible. Pigs and feeders were weighed on day (d) 0, 7, 14, 21 and 26 to determine the growth performance criteria of average daily gain (ADG), average daily food intake (ADFI), and feed conversion (F/G).

On d 27, one pig per pen was transported to the KSU meats lab for humane slaughter and collection of carcass data. Hot carcass weight was collected immediately after evisceration. First-rib, tenth-rib, last-rib, and last-lumbar backfat depth, as well as loin eye area at the 10^{th}-rib, were collected from the right half of each carcass 24-h postmortem. Additionally, each carcass was evaluated for loin muscle color at the 10^{th}-rib with a Hunter MiniScan instrument to measure CIE L*, a* and b*, known color parameters relating to brightness, red-to-green coloration and yellow-to-blue coloration, as known in the art. That was performed after 30 minutes of bloom time, the time to oxygenate myoglobin thereby obtaining maximal pink or red coloration, for each loin muscle surface.

In the agricultural arts, certain carcass and meat parameters or characteristics are monitored for economic benefit, not only from the standpoint of feed conversion efficiency but also for consumer preference and appeal. Some of the characteristics of interest to a hog producer include ADG, ADFI, fat amounts, meat color and so on. Such carcass and meat characteristics can lead to improvements in the net profit/pig. The US Department of Agriculture provides for grading of pork carcasses. Also, see, Buhr & DiPietre (1977) Front end guidance for value-added networks. National Pork Producers Council, Des Moines, Iowa; and National Pork Board (2000) Pork composition and quality assessment procedures. Berg, ed. Des Moines, Iowa.

Data were analyzed as a randomized complete block design using the PROC MIXED procedure of SAS with pen as the experimental unit. Linear and quadratic polynomial contrasts were used to determine the effect of increasing astaxanthin.

The source of natural astaxanthin used in the example was Phaffia rhodozyma which is commercially available under the trade name Aquasta® containing 10,000 ppm astaxanthin, by weight.

TABLE 1
Composition of Experimental Diets^{a}

| | Added Dietary Astaxanthin, ppm | | | |
|---|---|---|---|---|
| | 0 | 5 | 10 | 20 |
| Ingredient, % | | | | |
| Corn | 85.40 | 85.40 | 85.40 | 85.40 |
| Soybean meal, 46.5% CP | 12.44 | 12.44 | 12.44 | 12.44 |
| Monocalcium P, 21% P | 0.45 | 0.45 | 0.45 | 0.45 |
| Limestone | 0.85 | 0.85 | 0.85 | 0.85 |
| Salt | 0.35 | 0.35 | 0.35 | 0.35 |
| L-lysine HCl | 0.15 | 0.15 | 0.15 | 0.15 |
| Vitamin premix | 0.08 | 0.08 | 0.08 | 0.08 |
| Trace mineral premix | 0.08 | 0.08 | 0.08 | 0.08 |
| Corn starch | 0.20 | 0.15 | 0.10 | — |
| Aquasta ® (10,000 ppm) | — | 0.05 | 0.10 | 0.20 |
| TOTAL | 100.00 | 100.00 | 100.00 | 100.00 |
| Diet cost, $/100 pounds^{b} | 7.25 | 7.70 | 8.15 | 9.05 |
| Calculated analysis | | | | |
| Total lysine, % | 0.72 | 0.72 | 0.72 | 0.72 |
| True digestible amino | | | | |
| acids | | | | |
| Lysine, % | 0.63 | 0.63 | 0.63 | 0.63 |
| Isoleucine:lysine ratio, % | 71 | 71 | 71 | 71 |
| Leucine:lysine ratio, % | 188 | 188 | 188 | 188 |
| Methionine:lysine ratio, | 33 | 33 | 33 | 33 |
| % | | | | |
| Met & Cys:lysine ratio, | 68 | 68 | 68 | 68 |
| % | | | | |
| Threonine:lysine ratio, % | 64 | 64 | 64 | 64 |
| Tryptophan:lysine ratio, | 18 | 18 | 18 | 18 |
| % | | | | |
| Valine:lysine ratio, % | 85 | 85 | 85 | 85 |
| Protein, % | 13.2 | 13.2 | 13.2 | 13.2 |
| ME, kcal/lb | 1,522 | 1,522 | 1,522 | 1,522 |
| TID lysine:ME ratio, | 1.88 | 1.88 | 1.88 | 1.88 |
| g/Mcal | | | | |
| Ca, % | 0.47 | 0.47 | 0.47 | 0.47 |
| P, % | 0.42 | 0.42 | 0.42 | 0.42 |
| Available P, % | 0.15 | 0.15 | 0.15 | 0.15 |
| Analyzed astaxanthin | 0.8 | 4.8 | 9.5 | 19.8 |
| concentration, ppm | | | | |

^{a}Experimental diets fed for 26 d before slaughter.
^{b}Ingredient prices used to determine the diet cost were: corn, $118/ton; soybean meal, $207/ton; Monocalcium P, $332/ton; Limestone, $30/ton; Salt, $53/ton; L-lysine HCl, $1800/ton; Processing and delivery, $12/ton; and Astaxanthin, $0.0022/mg, in US dollars.

TABLE 2
Growth Performance and Carcass Characteristics of Pigs Fed Astaxanthin^{a}

| | | | | | | P < | | |
|---|---|---|---|---|---|---|---|---|
| | Astaxanthin, ppm | | | | SE | Control vs | | |
| Item | 0 | 5 | 10 | 20 | Mean | Asta | Linear | Quad |
| Growth Performance, | | | | | | | | |
| D 0 to 26 | | | | | | | | |
| Initial Wt., lb | 215 | 215 | 215 | 215 | 2.90 | — | — | — |
| ADG, lb | 2.11 | 2.23 | 2.03 | 1.99 | 0.12 | — | — | — |
| ADFI, lb | 6.67 | 6.76 | 6.24 | 6.20 | 0.24 | — | 0.10 | — |
| F/G | 3.22 | 3.05 | 3.08 | 3.16 | 0.15 | — | — | — |
| Final Wt., lb | 270 | 273 | 268 | 267 | 3.81 | — | — | — |
| Feed $/lb gain | 0.23 | 0.23 | 0.25 | 0.29 | 0.01 | — | 0.01 | — |
| Feed $/pig | 12.57 | 13.55 | 13.23 | 14.59 | 0.51 | 0.06 | 0.02 | — |
| Carcass | | | | | | | | |
| Characteristics | | | | | | | | |
| Live Wt., lb | 271 | 273 | 271 | 270 | 3.95 | — | — | — |
| Hot Carcass Wt., lb | 192 | 192 | 191 | 189 | 3.38 | — | — | — |
| Yield, % | 71.0 | 70.6 | 70.6 | 70.3 | 0.64 | — | — | — |
| Backfat-1^{st} rib, in. | 1.50 | 1.40 | 1.33 | 1.41 | 0.06 | 0.07 | — | 0.06 |
| Backfat-10^{th} rib, in. | 0.82 | 0.65 | 0.65 | 0.70 | 0.07 | 0.06 | — | 0.10 |
| Backfat-Last rib, | 0.78 | 0.65 | 0.67 | 0.64 | 0.06 | 0.07 | — | — |
| in. | | | | | | | | |
| Backfat-Last | 0.71 | 0.60 | 0.56 | 0.57 | 0.05 | 0.04 | — | — |
| Lumbar, in. | | | | | | | | |
| Loin eye area, sq. | 7.33 | 7.72 | 7.58 | 7.28 | 0.30 | — | — | — |
| in. | | | | | | | | |
| Loin eye color | | | | | | | | |
| CIE L* | 60.3 | 55.3 | 58.9 | 56.2 | 1.42 | 0.06 | — | — |
| CIE a* | 9.4 | 10.1 | 8.2 | 10.3 | 0.31 | — | — | 0.02 |
| CIE b* | 15.8 | 14.8 | 14.4 | 15.1 | 0.47 | 0.08 | — | 0.06 |
| Standardized Fat- | 102 | 107 | 106 | 103 | 2.37 | — | — | 0.09 |
| Free Lean, lb | | | | | | | | |
| Fat-Free Lean, % | 53.2 | 55.6 | 55.5 | 54.5 | 1.04 | 0.09 | — | 0.10 |
| Economic | | | | | | | | |
| Implications | | | | | | | | |
| Estimated Carcass | 68.76 | 70.13 | 70.08 | 69.41 | 0.65 | — | — | — |
| Value, $/100 lb^{b} | | | | | | | | |
| Estimated Total | 128.37 | 131.79 | 130.98 | 130.41 | 2.40 | — | — | — |
| Carcass Value, $ | | | | | | | | |
| Estimated Net | — | 2.44 | 1.95 | 0.02 | 3.31 | — | — | — |
| profit/loss per pig | | | | | | | | |
| relative to Control, $ | | | | | | | | |

^{a}A total of 48 barrows (PIC, TR4 × C22), with 2 pigs per pen and 6 pens per treatment, were used for comparing the growth performance. Carcass data were obtained from 1 pig per pen for the determination of carcass characteristics.
^{b}From the Sep. 13, 2007 USDA National Daily Direct Negotiated Hog Purchase Matrix with adjustments for carcass weight differentials.

As can be seen from the data, the average daily feed intake tended to decrease with increasing level of astaxanthin, but the ADG or F/G were not significantly different from the control pigs. Pigs fed astaxanthin had significantly (p<0.10) less backfat depth measured at the first rib (FRBF), tenth rib (TRBF) and last rib (LRBF). Pigs fed 5 or 10 ppm astaxanthin had the greatest percent fat-free lean (PFFL) (p<0.10) with decreased percent fat-free lean observed at 0 or 20 ppm astaxanthin incorporation. With respect to loin eye color, significant changes (p<0.10) were observed in CIE L* and CIE b* values. Improved characteristics observed for pigs fed 5 or 10 ppm astaxanthin resulted in improvements in the net profit/pig.

All references cited herein are herein incorporated by reference in entirety.

We claim:

1. A composition comprising isolated astaxanthin obtained from a Phaffia rhodozyma, comprising at least 500 ppm astaxanthin per gram dry weight of yeast, in an amount that enhances a finishing hog carcass parameter.

2. The composition of claim 1, wherein said amount is 5-10 ppm astaxanthin.

3. The composition of claim 1, wherein said amount is 5 ppm astaxanthin.

4. The composition of claim 1, wherein said amount is 10 ppm astaxanthin.

5. The composition of claim 1, wherein said parameter is back fat depth at the first rib.

6. The composition of claim 1, wherein said parameter is back fat depth at the tenth rib.

7. The composition of claim 1, wherein said parameter is back fat depth at the last rib.

8. The composition of claim 1, wherein said parameter is percent fat free lean.

9. The composition of claim 1, wherein said parameter is loin eye color.
